SEQUENCE LISTING (64 sequences)

SEQ ID NO: 1 - DNA, 58 bp, Artificial Sequence
  source: /note=“Description of Artificial Sequence Synthetic primer”
aatgatacggcgaccaccgagatctacactctttccctacacgacgctcttccgatct

SEQ ID NO: 2 - DNA, 23 bp, Artificial Sequence
  source: /note=“Description of Artificial Sequence Synthetic primer”
cactggatttagagtctctcagc

SEQ ID NO: 3 - DNA, 87 bp, Artificial Sequence
  source: /note=“Description of Artificial Sequence Synthetic primer”
  modified_base (65)..(67): a, c, t, g, unknown or other
caagcagaagacggcatacgagattggtcagtgactggagttcagacgtgtgctcttccgatctnnngctggtacacggcagggtca

SEQ ID NO: 4 - DNA, 20 bp, Artificial Sequence
  source: /note=“Description of Artificial Sequence Synthetic primer”
tgcttctgatggctcaaaca

SEQ ID NO: 5 - DNA, 90 bp, Artificial Sequence
  source: /note=“Description of Artificial Sequence Synthetic primer”
  modified_base (65)..(70): a, c, t, g, unknown or other
caagcagaagacggcatacgagatcactgtgtgactggagttcagacgtgtgctcttccgatctnnnnnncagcgacctcgggtgggaac

SEQ ID NO: 6 - protein, 8 aa, Artificial Sequence
  source: /note=“Description of Artificial Sequence Synthetic peptide”
ASSPGQDN

SEQ ID NO: 7 - protein, 7 aa, Artificial Sequence
  source: /note=“Description of Artificial Sequence Synthetic peptide”
AWGQGGN

SEQ ID NO: 8 - protein, 10 aa, Artificial Sequence
  source: /note=“Description of Artificial Sequence Synthetic peptide”
AWSPSGTGGR

SEQ ID NO: 9 - protein, 7 aa, Artificial Sequence
  source: /note=“Description of Artificial Sequence Synthetic peptide”
ASSLQSS

SEQ ID NO: 10 - protein, 9 aa, Artificial Sequence
  source: /note=“Description of Artificial Sequence Synthetic peptide”
ASSLVWEKQ

SEQ ID NO: 11 - protein, 4 aa, Artificial Sequence
  source: /note=“Description of Artificial Sequence Synthetic peptide”
ASGG

SEQ ID NO: 12 - protein, 8 aa, Artificial Sequence
  source: /note=“Description of Artificial Sequence Synthetic peptide”
ASSFGATS

SEQ ID NO: 13 - protein, 8 aa, Artificial Sequence
  source: /note=“Description of Artificial Sequence Synthetic peptide”
ASSLGQGN

SEQ ID NO: 14 - protein, 7 aa, Artificial Sequence
  source: /note=“Description of Artificial Sequence Synthetic peptide”
ASSLGVA

SEQ ID NO: 15 - DNA, 24 bp, Artificial Sequence
  source: /note=“Description of Artificial Sequence Synthetic oligonucleotide”
gccagctctccgggacaggataat

SEQ ID NO: 16 - DNA, 21 bp, Artificial Sequence
  source: /note=“Description of Artificial Sequence Synthetic oligonucleotide”
gcctggggacagggaggcaac

SEQ ID NO: 17 - DNA, 30 bp, Artificial Sequence
  source: /note=“Description of Artificial Sequence Synthetic oligonucleotide”
gcctggagtccttccgggactggggggagg

SEQ ID NO: 18 - DNA, 21 bp, Artificial Sequence
  source: /note=“Description of Artificial Sequence Synthetic oligonucleotide”
gcaagcagcttgcagtctagt

SEQ ID NO: 19 - DNA, 27 bp, Artificial Sequence
  source: /note=“Description of Artificial Sequence Synthetic oligonucleotide”
gccagcagtttggtctgggagaagcaa

SEQ ID NO: 20 - DNA, 12 bp, Artificial Sequence
  source: /note=“Description of Artificial Sequence Synthetic oligonucleotide”
gccagcggtgga

SEQ ID NO: 21 - DNA, 24 bp, Artificial Sequence
  source: /note=“Description of Artificial Sequence Synthetic oligonucleotide”
gccagcagtttcggggcaactagt

SEQ ID NO: 22 - DNA, 24 bp, Artificial Sequence
  source: /note=“Description of Artificial Sequence Synthetic oligonucleotide”
gccagctctctaggacaggggaac

SEQ ID NO: 23 - DNA, 21 bp, Artificial Sequence
  source: /note=“Description of Artificial Sequence Synthetic oligonucleotide”
gccagctctctcggtgttgca

SEQ ID NO: 24 - DNA, 50 bp, Homo sapiens
gtgggcttctgggtgtttttgactgactaagaaacactgtgggatggata

SEQ ID NO: 25 - DNA, 49 bp, Homo sapiens
  modified_base (1)..(1): a, c, t, g, unknown or other
naaacactgtgatactcacgggaggaggaaacaaactcacctttgggac

SEQ ID NO: 26 - DNA, 28 bp, Homo sapiens
aggcactcagctaaaagtggaactcaat

SEQ ID NO: 27 - DNA, 34 bp, Homo sapiens
atccagaagcctgaccctgccgtgtaccagctaa

SEQ ID NO: 28 - DNA, 40 bp, Artificial Sequence
  source: /note=“Description of Artificial Sequence Synthetic oligonucleotide”
tactgcacctgcagtgcagacaattctggaaatacgctct

SEQ ID NO: 29 - DNA, 40 bp, Artificial Sequence
  source: /note=“Description of Artificial Sequence Synthetic oligonucleotide”
tactgcacctgcagtgcagatgcagaaacgctgtattttg

SEQ ID NO: 30 - DNA, 40 bp, Artificial Sequence
  source: /note=“Description of Artificial Sequence Synthetic oligonucleotide”
ttttgtgccagcagccaaggacagtatgaacagtacttcg

SEQ ID NO: 31 - protein, 13 aa, Artificial Sequence
  source: /note=“Description of Artificial Sequence Synthetic peptide”
YCTCSADNSGNTL

SEQ ID NO: 32 - protein, 13 aa, Artificial Sequence
  source: /note=“Description of Artificial Sequence Synthetic peptide”
YCTCSADAETLYF

SEQ ID NO: 33 - protein, 13 aa, Artificial Sequence
  source: /note=“Description of Artificial Sequence Synthetic peptide”
FCASSQGQYEQYF

SEQ ID NO: 34 - DNA, 35 bp, Artificial Sequence
  source: /note=“Description of Artificial Sequence Synthetic oligonucleotide”
  CDS (3)..(35)
tggatgacactgccacctacatctgtgtggtgggc

SEQ ID NO: 35 - protein, 11 aa, Artificial Sequence
  source: /note=“Description of Artificial Sequence Synthetic peptide”
DDTATYICVVG

SEQ ID NO: 36 - DNA, 33 bp, Artificial Sequence
  source: /note=“Description of Artificial Sequence Synthetic oligonucleotide”
  CDS (1)..(33)
gatagaggttcagccttagggaggctgcatttt

SEQ ID NO: 37 - protein, 11 aa, Artificial Sequence
  source: /note=“Description of Artificial Sequence Synthetic peptide”
DRGSALGRLHF

SEQ ID NO: 38 - protein, 9 aa, Artificial Sequence
  source: /note=“Description of Artificial Sequence Synthetic peptide”
ICVVGDRGS

SEQ ID NO: 39 - protein, 9 aa, Artificial Sequence
  source: /note=“Description of Artificial Sequence Synthetic peptide”
ICVVIDRGS

SEQ ID NO: 40 - protein, 9 aa, Artificial Sequence
  source: /note=“Description of Artificial Sequence Synthetic peptide”
ICVVDRGSA

SEQ ID NO: 41 - protein, 9 aa, Artificial Sequence
  source: /note=“Description of Artificial Sequence Synthetic peptide”
ICVVVDRGS

SEQ ID NO: 42 - protein, 9 aa, Artificial Sequence
  source: /note=“Description of Artificial Sequence Synthetic peptide”
ICVVADRGS

SEQ ID NO: 43 - protein, 9 aa, Artificial Sequence
  source: /note=“Description of Artificial Sequence Synthetic peptide”
ICVVGAEVQ

SEQ ID NO: 44 - protein, 9 aa, Artificial Sequence
  source: /note=“Description of Artificial Sequence Synthetic peptide”
ICVVGHRGS

SEQ ID NO: 45 - protein, 9 aa, Artificial Sequence
  source: /note=“Description of Artificial Sequence Synthetic peptide”
ICVVGNRGS

SEQ ID NO: 46 - protein, 9 aa, Artificial Sequence
  source: /note=“Description of Artificial Sequence Synthetic peptide”
ICVVGAGGS

SEQ ID NO: 47 - protein, 9 aa, Artificial Sequence
  source: /note=“Description of Artificial Sequence Synthetic peptide”
ICVVGERFS

SEQ ID NO: 48 - protein, 9 aa, Artificial Sequence
  source: /note=“Description of Artificial Sequence Synthetic peptide”
ICVGTERLH

SEQ ID NO: 49 - protein, 9 aa, Artificial Sequence
  source: /note=“Description of Artificial Sequence Synthetic peptide”
ICVVGRGSA

SEQ ID NO: 50 - protein, 9 aa, Artificial Sequence
  source: /note=“Description of Artificial Sequence Synthetic peptide”
SVWWGYRGS

SEQ ID NO: 51 - protein, 9 aa, Artificial Sequence
  source: /note=“Description of Artificial Sequence Synthetic peptide”
ICEVGDRGS

SEQ ID NO: 52 - protein, 9 aa, Artificial Sequence
  source: /note=“Description of Artificial Sequence Synthetic peptide”
SVQNRGSAL

SEQ ID NO: 53 - protein, 9 aa, Artificial Sequence
  source: /note=“Description of Artificial Sequence Synthetic peptide”
ICVVGDRGP

SEQ ID NO: 54 - protein, 9 aa, Artificial Sequence
  source: /note=“Description of Artificial Sequence Synthetic peptide”
ICVVGGERF

SEQ ID NO: 55 - protein, 9 aa, Artificial Sequence
  source: /note=“Description of Artificial Sequence Synthetic peptide”
ICMVGDRGS

SEQ ID NO: 56 - protein, 9 aa, Artificial Sequence
  source: /note=“Description of Artificial Sequence Synthetic peptide”
ICVVGARGS

SEQ ID NO: 57 - protein, 9 aa, Artificial Sequence
  source: /note=“Description of Artificial Sequence Synthetic peptide”
ICVVGARRG

SEQ ID NO: 58 - protein, 9 aa, Artificial Sequence
  source: /note=“Description of Artificial Sequence Synthetic peptide”
ICVVGGRGS

SEQ ID NO: 59 - protein, 9 aa, Artificial Sequence
  source: /note=“Description of Artificial Sequence Synthetic peptide”
VCVVGDRGS

SEQ ID NO: 60 - DNA, 30 bp, Artificial Sequence
  source: /note=“Description of Artificial Sequence Synthetic oligonucleotide”
  CDS (3)..(29)
gtgtggtgggcgatagaggttcagccttag

SEQ ID NO: 61 - protein, 9 aa, Artificial Sequence
  source: /note=“Description of Artificial Sequence Synthetic peptide”
VVGDRGSAL

SEQ ID NO: 62 - DNA, 30 bp, Artificial Sequence
  source: /note=“Description of Artificial Sequence Synthetic oligonucleotide”
  CDS (3)..(29)
gtgtggtgggggatagaggttcagccttag

SEQ ID NO: 63 - DNA, 30 bp, Artificial Sequence
  source: /note=“Description of Artificial Sequence Synthetic oligonucleotide”
  CDS (3)..(29)
gtgtggtgggagatagaggttcagccttag

SEQ ID NO: 64 - DNA, 30 bp, Artificial Sequence
  source: /note=“Description of Artificial Sequence Synthetic oligonucleotide”
  CDS (3)..(29)
gtgtggtgggtgatagaggttcagccttag

(19) United States
(12) Patent Application Publication
(10) Pub. No.: US 2019/0055607 A1
(43) Pub. Date: Feb. 21, 2019

(54) METHODS AND COMPOSITIONS FOR DETERMINING SPECIFIC TCR AND BCR CHAIN PAIRINGS

(71) Applicant: GIRIHLET INC., Oakland, CA (US)

(72) Inventor: Anitha JAYAPRAKASH, OAKLAND, CA (US)

(21) Appl. No.: 16/179,869

(22) Filed: Nov. 2, 2018

Related U.S. Application Data
(63) Continuation of application No. PCT/US2017/031444, filed on May 5, 2017.
(60) Provisional application No. 62/333,110, filed on May 6, 2016.

(51) Int. Cl. C12Q 1/6883

(52) U.S. Cl. CPC C12Q 1/6883; C12Q 2600/16

(57) ABSTRACT

Provided are methods for determining TCR α/γ and TCR β/δ chain pairings in T cell(s) of interest within a population of T cells, comprising a combination of selecting for the T cell(s) of interest based on the TCR α/δ or TCR β/δ chain followed by sequencing of the counterpart TCR β/δ or TCR α/γ chain, respectively.

### CROSS-REFERENCE TO RELATED APPLICATIONS

This application is a continuation of International Patent Application No. PCT/US2017/031444 with a filing date of May 5, 2017, designating the United States, now pending, and further claims priority to U.S. provisional application 62/333,110 with a filing date of May 6, 2016. The content of the aforementioned applications, including any intervening amendments thereto, are incorporated herein by reference.

### BACKGROUND

### Field

The invention relates to methods for identifying T cell and B cell receptor chain pairings, and the use of such pairs in diagnostic and therapeutic applications.

### Description of the Related Art

T cells are the central players in the immune system with effector functions to kill infected (or abnormal) cells, and regulate other T cells and B-cells. The T cell receptor (TCR) expressed by T cells allows them to recognize antigenic peptides presented to them by the major histocompatibility complexes (MHC) on the surfaces of cells (MHC class I on all cells, MHC class II on professional antigen presenting cells). All TCRs are heterodimers of receptor pairs: the majority of T cells, called α/β T cells, express the α-β receptor pair; the rest (<10%), called γ/δ T cells, express the γ-δ pair.

Each chain of the TCR is encoded by one of the four multigene families (α, β, γ, δ). The organization of the TCR loci in mice and humans includes an array of Variable (V), Diversity (D, present only in β and δ), Joining (J) and Constant (C) gene segments. The assembly of these genes is what generates the diversity of the T cell repertoire: one pair of VDJC (in β, δ) or VJC (in α, γ) is selected from one allele in each developing thymocyte, and this pair then remains unique to that cell and its progeny.

Each recombined TCR possesses unique antigen specificity, determined by the structure of the antigen-binding site formed by the complementarity determining regions (CDR) of the α and β chains in the case of αβ T cells, or the γ and δ chains on case of γδ T cells. The TCR α and γ chains are generated by VJ recombination, whereas the β and δ chains occur by VDJ recombination. Of the several complementarity determining regions that determine the antigen-specificity of the TCR, the CDR3 (the junction of V-J) is the most important for peptide/MHC recognition. For the β and γ TCRs, one of a few ‘diversity’ (D) regions is interposed between the V and the J. The diversity in CDR3 is generated by the choice of V, D (for β, γ) and J, and by deletions and non-templated insertions.

Enzymes that are involved in the recombination process include the RAG (Recombination Activating Genes) proteins that recognize Recombination Signal Sequences (RSS). RSS flank each gene segment and consist of a heptamer (CACAGTG) and a conserved nonamer (ACAAAAACC), which is separated by a spacer of 12 or 23 base pairs (bp). The recombination process obeys the 12-23 rule, where a recognition signal with a 12-nucleotide spacer can only recombine with another having a 23-nucleotide spacer (FIG. 2). The frequency of recombination of certain V (D) J pairs can be greatly reduced, depending on the extent of alterations in the RSS. As noted above, the specificity of the TCR repertoire is determined by the variable junction of the V and J, called the CDR3. By combinatorial joining and random addition of nucleotides, a large number, in the tens of millions, of TCRs can be formed but not all of them make the mature functional repertoire. Thymic selection is thought to reduce the repertoire to ˜10¹³ possible combinations (in mice to ˜1-2 10⁸ T cells) (Casrouge et al., Size estimate of the αβ TCR repertoire of naive mouse splenocytes. J. Immunol. 11:5782-5787 (2000). But each T cell expresses only one α and one β chain, or one γ and one δ chain.

Next-generation sequencing of immunoglobulin variable region and T-cell receptor repertoires is providing information that is key to understanding adaptive immune responses and to diagnostic and therapeutic applications. (Reddy et al. Monoclonal antibodies isolated without screening by analyzing the variable-gene repertoire of plasma cells. Nat. Biotechnol. 28:965-969 (2010); Wu et al. Focused evolution of HIV-1 neutralizing antibodies revealed by structures and deep sequencing. Science 333:1593-1602 (2011); Ippolito et al. Antibody repertoires in humanized NOD-scid-IL2R gamma (null) mice and human B cells reveals human-like diversification and tolerance checkpoints in the mouse. PLoS ONE 7, e35497 (2012); Reddy & Georgiou Systems analysis of adaptive immunity by utilization of high-throughput technologies. Curr. Opin. Biotechnol. 22:584-589 (2011); Weinstein et al. High-throughput sequencing of the zebrafish antibody repertoire. Science 324:807-810 (2009); Benichou et al. Rep-Seq: uncovering the immunological repertoire through next-generation sequencing. Immunology 135:183-191 (2012)). However, existing immune repertoire sequencing technologies yield data on only one of the two chains of immune receptors and thus cannot provide information about the identity of immune receptor pairs encoded by individual B or T lymphocytes. (Wu et al, supra; 7. Fischer, N. Sequencing antibody repertoires: the next generation. MAbs 3:17-20 (2011); Wilson & Andrews Tools to therapeutically harness the human antibody response. Nat. Rev. Immunol. 12:709-719 (2012).

With respect to analyzing T cell repertoires in particular, conventional approaches measure bulk populations of alpha and beta receptors, but provide no information regarding which alphas and betas occur together in which cell. Several techniques have been described for detection or sequencing of genomic DNA or cDNA from single cells; however, all are limited by low efficiency or low cell throughput (<200-500 cells) and require fabrication and operation of complicated microfluidic devices. Marcus et al. Microfluidic single-cell mRNA isolation and analysis. Anal. Chem. 78:3084-3089 (2006); White et al. High-throughput microfluidic single-cell RT-qPCR. Proc. Natl. Acad. Sci. USA 108:13999-14004 (2011); Furutani et al. Detection of expressed gene in isolated single cells in microchambers by a novel hot cell-direct RT-PCR method. Analyst 137:2951-2957 (2012); Turchaninova et al. Pairing of T-cell receptor chains via emulsion PCR. Eur. J. Immunol. 43:2507-2515 (2013).

Chudakov and coworkers, for example, recently reported the use of one-pot cell encapsulation within water-in-oil emulsions, achieving cell lysis by heating at 65° C. concomitant with reverse transcription of the genes encoding T cell receptor α (TCRα) and TCRβ and linking by overlap-extension PCR to determine the TCRα-TCRβ pairings, albeit only for TCRβV7 and with a very low efficiency (approximately 700 TCRα-TCRβ pairs recovered from 8×10⁶ peripheral blood mononuclear cells. Turchaninova et al., supra. Similarly, DeKosky et al. have described single cell emulsification combined with emulsion linkage RT PCR, tagging with a random barcode and performing deep sequencing (millions of cells). DeKosky et al., In-depth determination and analysis of the human paired heavy- and light-chain antibody repertoire Nature Medicine (2014). Unfortunately, however, three sequencing reactions and in silico assembly are needed to determine the sequence of the complete linked VH-VL, making this approach quite burdensome and tedious in anything other than an academic setting.

Accordingly, there is clearly still a need for methods for determining specific pairing of alpha and beta T cell receptors, at least for identifying medically relevant and/or actionable members of the TCR repertoire. The methods described herein help meet these and other needs.

### BRIEF SUMMARY

The present invention solves the foregoing problems in the prior art by providing a method for rapidly and accurately determining the α and β chain or γ and δ chain pairings in T cells of interest (e.g. clinically-relevant T cells) within a population of T cells, without having to individually analyze all of the T cells. The invention derives in part from the recognition that the α/γ and β/δ chains are associated with each other across a given population of T cells in a more restricted manner than previously recognized, and accordingly pairings can be identified more efficiently by first selecting for either the TCR α/γ or β/δ chain in the T cell(s) of interest based on the variable domain, e.g., at least a portion of the complementarity determining region, and then sequencing the counterpart TCR β/δ or α/γ chain, respectively, of the selected T cell(s). The inventive method thus comprises a novel combination of T cell sorting based on probes directed to at least a portion of the CDR from one of the TCR α/γ or β/δ chain, e.g., the CDR3 region, in the T cell(s) of interest, followed by sequencing of the associated TCR β/δ or α/γ chain, respectively, enabling one to more rapidly and efficiently determine pairings and identify clones that might be responsive to infections or tumors, or responsible for allergic or autoimmune responses.

In one aspect, the invention provides a method for determining TCR α/γ and β/δ chain pairings in a population of T cells comprising a) contacting said population of T cells with a plurality of labelled probes directed to either the TCR α/γ chain or TCR β/δ chain of a T cell(s) of interest; b) sorting said population of T cells based on binding of said plurality of labelled probes to select for said T cell(s) of interest; and c) sequencing the counterpart TCR β/δ or TCR α/γ chain, respectively, in the selected T cell(s). In some embodiments, the labelled probes target at least a portion of the variable domain, e.g., all or a portion of the V and/or J regions (and/or D regions in TCR β/δ), more preferably at least a portion of the complementarity determining region, and still more preferably at least a portion of the CDR3 region in the TCR α/γ chain or TCR β/δ chain.

A variety of sorting techniques can be performed to select for the T cell(s) of interest based on binding of the labelled probes to either of the α/γ or β/δ chains, including radioactive assays and non-radioactive assays based on optical methods, e.g., fluorescence, phosphorescence, chemoluminescence, electrochemoluminescence, fluorescence polarization, fluorescence resonance energy transfer or surface plasmon resonance. In preferred embodiments, sorting is done by flow cytometry.

In some embodiments, antibodies directed against V, J or CDR3 segments can be used to tag the cells, which can then be sorted by flow cytometry. In alternative embodiments, nucleic acid probes are used, and more preferably RNA probes. In specific embodiments, live cell RNA detection is employed using, e.g., labelled sequence-specific RNA probes such as the Smartflare™ RNA Detection Technology available from EMD Millipore (see, e.g. McClellan et al. mRNA detection in living cells: A next generation cancer stem cell identification technique Methods 82:47-54 (2015)); or alternatively the PrimeFlow™ RNA assay available from Affymetrix Inc. (Henning et al. Measurement of Low-Abundance Intracellular mRNA Using Amplified FISH Staining and Image-Based Flow Cytometry. Curr Protoc Cytom. 76:7.46.1-8 (2016).

Similarly, a variety of sequencing techniques can be performed to identify the counterpart β or α chain in the T cell(s) of interest selected by way of the sorting step, generally comprising amplification of the TCR region of the selected T cells via the polymerase chain reaction or variations thereof. In preferred embodiments, the sequencing step comprises the nested PCR technique described in International PCT Application No. PCT/US15/62018, wherein primers directed to the constant (C) region along with universal primers are preferably employed to capture TCR mRNAs in fragmented mRNA. Alternatively, the sequencing step comprises the nested PCR technique described in U.S. Patent Publication 2016/0034637, wherein primers directed to the variable (V) region and to the joining (J) or C region are preferably employed. More conventional methodologies can also be used.

In one embodiment, T cell(s) of interest (e.g. having clinically relevant CDR3) may be identified by an initial bulk sequencing of a sample taken from the population of T cells, and evaluating relative α/γ and β/δ chain abundance in comparison with a sample indicative of T cell expansion, e.g., comparison of a disease-state sample from a subject against either an earlier sample from the same subject, or a baseline control sample, or a comparison between tissue- or lymph node-derived T cells and circulating blood-derived T cells in said subject. Alternatively, comparison may be made with an appropriate population database that contains the diversity of TCR repertoires across different ethnic, gender and age groups.

A preferred indicator is the percentage or fractional change in abundance and CDR3s of potential interest may be identified in this way. A CDR3 showing a large relative change in population (as a fraction) compared to the original state can be used to design labelled probes for use in the subject methods, i.e. probes directed to the identified CDR3 in the α/γ and β/δ chain, followed by sequencing of the counterpart β/δ or α/γ chain, respectively. Notably, the corresponding partner α/γ and β/δ chain might not show the same large change if it is abundant in the original sample, making the relative change not significant.

In another embodiment, the method further comprises the initial step of obtaining a biological sample comprising said population of T cells from a subject. In some embodiments, the subject exhibits a disease or disease symptoms. In one embodiment, the subject is a tumor-bearing, and the T cells are tumor-infiltrating lymphocytes. In another embodiment, the subject is suffering from an infection. In another embodiment, the subject is suffering from an allergic condition. In another embodiment, the subject is suffering from an autoimmune disorder. In preferred embodiments, the subject is human.

In some embodiments, the biological sample is a body fluid sample and/or tissue sample. In some embodiments, the biological sample is selected from the group consisting of blood, plasma, serum, bone marrow, semen, vaginal secretions, urine, amniotic fluid, cerebrospinal fluid, synovial fluid and biopsy tissue samples, including from infection and/or tumor locations.

As the skilled artisan will readily appreciate, the subject methods may also find advantageous use in identifying and analyzing B cell receptors, wherein the light chain and heavy chain molecules correspond to the TCR chains in the T cell repertoire. In manner similar to that described for T cells above, one can identify interesting members of the heavy and light chain by sequencing the RNA and use specific probes to the variable parts of a light or heavy chain(s) to select for B cell clones of interest, and then sequence the counterpart heavy or light chain, respectively, in the selected B cells.

### FIGURES

FIG. 1 shows the actual distribution of beta chain VJ pairs in a human (thick dark line), along with expected frequencies (thin light line) based on individual V and J frequencies in the bottom/left graph. V and J pairings are not “predictable”. Similarly, alpha, beta pairings cannot be predicted from bulk measurements.

FIG. 2 illustrates that abundant VJ pairings follow a zipf (1/f) law while the tail follows an exponential decay. Large changes in the abundant pairings will not appear to be significant and their impact on function of the immune system will be limited. A significant change in either of the individual alpha or beta chain frequencies can be identified from bulk and the counterpart chain identified following the methods disclosed herein.

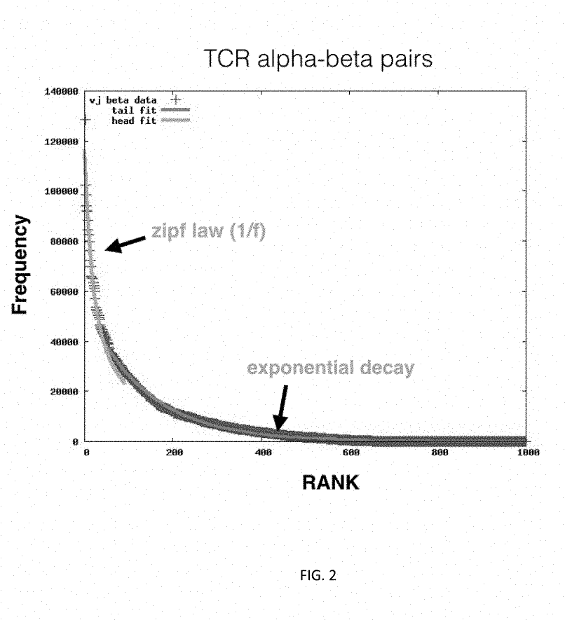

FIG. 3 illustrates a table showing data from TCR repertoire sequencing carried out on RNA isolated from 2 mL of human blood.

FIG. 4 illustrates a table showing data from TCR repertoire sequencing carried out on RNA isolated from 2 mL of human blood (SEQ ID NOs 24-27, respectively, in order of appearance).

FIG. 5 illustrates a table showing output of an analytical pipeline that tracks TCR clonality and annotations. CDR3 oligonucleotides disclosed as 28-30, respectively, in order of appearance, and CDR3 peptides disclosed as 31-33, respectively, in order of appearance.

FIG. 6 illustrates a table showing frequency of a CDR3 sequence in three strains of mice. FIG. 6 discloses SEQ ID NOs 34-37, respectively, in order of appearance. Columns 1-4 disclose the sequence “ICVVGDRGS” as SEQ ID NO: 38. Column 5 discloses SEQ ID NOs 39, 40, 41, 38, 42, 41, 38, 42, 42, 38, 42, 42, and 42, respectively, in order of appearance. Column 6 discloses SEQ ID NOs 41, 41, 43, 44, 38, 42, 45, 41, 45, 41, 38, 45, and 41, respectively, in order of appearance. Column 7 discloses SEQ ID NOs 46, 56, 46, 42, 38, 42, 41, 42, 47, 42, 57, 41, and 48, respectively, in order of appearance. Column 8 discloses SEQ ID NOs 41, 38, 49, 42, 41, 38, 42, 38, 42, 42, 38, 41, and 50, respectively, in order of appearance. Column 9 discloses SEQ ID NOs 38, 38, 38, 41, 51, 41, 52, 53, 41, 54, 55, 42, and 38, respectively, in order of appearance. Column 10 discloses SEQ ID NOs 41, 38, 59, 41, 38, 45, 56, 38, 57, 58, 41, 49, and 59, respectively, in order of appearance. FIG. 6 also discloses SEQ ID NOs 60, 61, 62, 61, 63, 61, 64, and 61, respectively, in order of appearance.

### DETAILED DESCRIPTION

Each T cell expresses an α and a β chain, or a γ and a δ chain, which together determine the specificity of the antigen (presented by the MHC) that the T cell can recognize. The α and β chain or γ and a δ chain combinations in each cell are difficult to identify, since many millions of cells need to be individually interrogated. Provided herein are methods for identifying the pairs in a restricted set of cells, based on bulk data, which simplifies the analysis immensely and ensures timely, inexpensive identification of clones of interest for diagnostic and therapeutic use.

In the following description, certain specific details are set forth in order to provide a thorough understanding of various embodiments of the disclosure. However, one skilled in the art will understand that the disclosure may be practiced without these details.

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

As used herein the term “about” refers to ±10%.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Labelled probes finding advantageous use in the inventive methods may comprise any of the detecting means specific ally disclosed herein, as well as any other detecting means known in the art, including, e.g.: fluorophores; biotin and biotinylation radioisotopes; affinity peptide tags (e.g., His tags, myc tags, FLAG tags, and the like); which may be conjugated to, linked to, or otherwise associated with (such as by either a covalent or a non-covalent linkage or bond) a nucleic acid, polypeptide, antibody, and/or a test binding partner (e.g., an antigen).

Similarly, any means for detecting the interaction between the labelled probe and the CDR region of the TCRα or β chain of the T cells may be employed in accordance with the sorting step disclosed and claimed herein. Exemplary such means include radioactive assays and non-radioactive assays based on optical methods, e.g., fluorescence, phosphorescence, chemoluminescence, electrochemolumninescence, fluorescence polarization, fluorescence resonance energy transfer or surface plasmon resonance. In some embodiments, the detection means comprise flow cytometry; magnetic-activated cell sorting (MAGS); florescence assisted cell sorting (FACS); immunohistochemistry; column and/or affinity chromatography or separations; sedimentation methodologies (e.g., centrifugation); immunoprecipitation; florescence resonance energy transfer (FRET) assays; affinity chromatography; and the like. In preferred embodiments, the sorting step comprises florescence assisted cell sorting (FACS).

In some embodiments, antibodies directed against V, J or CDR3 segments can be used to tag the cells, which can then be sorted by flow cytometry. In alternative embodiments, nucleic acid probes are used, and more preferably RNA probes. In specific embodiments, live cell RNA detection is employed using, e.g., labelled sequence-specific RNA probes such as the Smartflare™ RNA Detection Technology available from EMD Millipore (see, e.g. McClellan et al. mRNA detection in living cells: A next generation cancer stem cell identification technique Methods 82:47-54 (2015)); or alternatively the PrimeFlow™ RNA assay available from Affymetrix Inc. (Henning et al. Measurement of Low-Abundance Intracellular mRNA Using Amplified FISH Staining and Image-Based Flow Cytometry. Curr Protoc Cytom. 76:7.46.1-8 (2016).

In one embodiment, the labelled probes comprise the Smartflare™ RNA Detection Technology available from EMD Millipore, using gold nanoparticles bound to sequence-specific oligonucleotide probes. In the presence of their targets, the probes fluoresce and the cells can be imaged and sorted using flow cytometry. Over time, the probes exit the cell through natural exocytosis, without adverse effects, thus enabling downstream assays on the same sample.

In another embodiment, labelled probes based on the PrimeFlow™ RNA Assay can be employed, using fluorescent in situ hybridization (FISH) techniques for simultaneous detection of up to three RNA transcripts in a single cell using a standard flow cytometer. Development of the latter assay is based upon Affymetrix® ViewRNA™ FISH assays, combining paired nucleic acid probe design with branched DNA (bDNA) signal amplification to detect gene expression at the single-cell level. The variable region-specific probe sets will typically contain 20-40 oligonucleotide pairs that hybridize to the target RNA transcript. Signal amplification is achieved through specific hybridization of adjacent oligonucleotide pairs to bDNA structures, formed by pre-amplifiers, amplifiers and fluorochrome-conjugated label probes, resulting in excellent specificity, low background and high signal-to-noise ratio.

The probes used in the SmartFlare™ or Primeflow™ assays may advantageously comprise i) standard DNA; ii) standard RNA; iii) Locked Nucleic Acid (LNA, available from Exiqon Inc.) (see e.g., Ishige et al., Locked nucleic acid probe in particular enhances Sanger sequencing sensitivity and improves diagnostic accuracy of high-resolution melting-based KRAS mutational analysis Clin Chim Acta. 981(16):30130-9 (2106)); iv) various modified single strand DNA or RNA such as SAMRS (available from Firebird Bio Inc.) (see, e.g., Glushakova et al., High-throughput Multiplexed xMAP Luminex Array Panel for Detection of Twenty TWO Medically Important Mosquito-borne Arboviruses based on Innovations in Synthetic Biology J Virol Methods 214:60-74 (2015)). To isolate specific T cell clones, we can use two RNA or DNA probes, using the Smartflare™ or Primeflow™ technology described above, directed against the CDR3 (α and β) to ensure precise identification of the clone.

As a preliminary step, the T cell(s) of interest (e.g. having clinically relevant CDR3) may be identified by bulk sequencing of a sample taken from the population of T cells, and evaluating relative α/γ and β/δ chain abundance in comparison with a sample indicative of T cell expansion, e.g., comparison of a disease-state sample from a subject against either an earlier sample from the same subject, or a baseline control sample, or a comparison between tissue- or lymph node-derived T cells and circulating blood-derived T cells in said subject. Comparisons of the repertoire can be made either between samples in a time-series of measurements or in comparison to a population level measure. Comparisons can also be carried out between T cells derived from diseased tissue/lymph nodes versus circulating blood levels. Notably, changes in abundant species will not impact immune function as much as changes in a rarer species. This follows from the kinetics of biochemical reaction determined by reactant concentrations. In an alpha-beta pair, one member might show significant change while the other might not, because it could be one of an abundant species. Accordingly, identifying individual CDR3 from the alpha and beta repertoires that have “changed” significantly is the key to identifying alpha-beta pairings in clones that are biologically relevant.

A variety of sequencing techniques can be performed in accordance with the inventive teachings herein. DNA sequencing techniques include classic dideoxy sequencing reactions (Sanger method) using labeled terminators or primers and gel separation in slab or capillary, sequencing-by-synthesis using reversibly terminated labeled nucleotides, pyrosequencing, allele specific hybridization to a library of labeled oligonucleotide probes, sequencing-by-synthesis using allele specific hybridization to a library of labeled clones that is followed by ligation, real time monitoring of the incorporation of labeled nucleotides during a polymerization step, and SOLiD (Life Technologies, Inc.), Ion Torrent™ (Life Technologies, Inc.), HiSeq™ and MiSeq™, SOLEXA™, SMRT™, nanopore, Genome Sequencer FLX™ (Roche), and Chemical-Sensitive Field Effect Transistor Array Sequencing (chemFET) sequencing. Additional analysis techniques include true single molecule sequencing (tSMS; Helicos True Single Molecule Sequencing) (Harris et al. (2008) Science 320:106-109), 454 Sequencing (Roche) (Margulies et al. (2005) Nature 437:376-380); and Arm-PCR or tem-PCR (Han et al. (2006) J. Clin. Micro. 44(11):4157-4162)

Prior to or as part of the contacting and/or sequencing steps, the nucleic acid component of the T cell population, e.g. the TCR mRNA, can be isolated, fragmented and optionally amplified using standard techniques and methodologies (Sambrook et al., MOLECULAR CLONING: A LABORATORY MANUAL, 2nd edition Cold Spring Harbor Laboratory Press, CSH, (1989) and Current Protocols (Genetics and Genomics; Molecular Biology; 2003-2013), both of which are incorporated herein by reference for all purposes).

Cell lysis is a commonly practiced method for the recovery of nucleic acids from within cells. In many cases, the cells are contacted with a lysis solution, commonly an alkaline solution comprising a detergent, or a solution of a lysis enzyme. Such lysis solutions typically contain salts, detergents and buffering agents, as well as other agents that one of skill would understand to use. After full and/or partial lysis, the nucleic acids are recovered from the lysis solution. In some embodiments, all solutions and equipment employed is RNAase free. Methods for RNAse decontamination and preparation of RNAse free solutions are well known to those of skill in the art and such methods can be readily applied as needed by one practicing the methods disclosed herein.

A variety of lysis solutions have been described and are known to those of skill in the art. Any of these well known lysis solutions can be employed with the present methods in order to isolate nucleic acids from a sample, in particular mRNA. Exemplary lysis solutions include those commercially available, such as those sold by INVITROGEN®, QIAGEN®, LIFE TECHNOLOGIES® and other manufacturers, as well as those which can be generated by one of skill in a laboratory setting. Lysis buffers are also well known and a variety of lysis buffers can be used in the disclosed methods, including for example those described in Molecular Cloning and Current Protocols, supra.

In some embodiments, the nucleic acids, including for example but not limited to mRNA, are isolated from a lysis buffer. Any of a variety of methods useful in the isolation of small quantities of nucleic acids are used by various embodiments of the disclosed methods. These include but are not limited to precipitation, gel filtration, density gradients and solid phase binding. In some embodiments, total RNA used in the methods of the present disclosure can also be obtained from simple extraction methods, such as, Trizol extraction. Total RNA samples used in the present invention may or may not be treated with DNases prior to cDNA generation.

Nucleic acid precipitation is a well know method for isolation that is known by those of skill in the art. A variety of solid phase binding methods are also known in the art including but not limited to solid phase binding methods that make use of solid phases in the form of beads (e.g., silica, magnetic), columns, membranes or any of a variety other physical forms known in the art. Substrates typically contain polyT tags, which bind to the polyA tail of the mRNA. Such substrates can include for example Ampure Beads form Beckman Coulter. In some embodiments, solid phases used in the disclosed methods reversibly bind nucleic acids. Examples of such solid phases include so-called “mixed-bed” solid phases are mixtures of at least two different solid phases, each of which has a capacity to nucleic acids under different solution conditions, and the ability and/or capacity to release the nucleic acid under different conditions; such as those described in U.S. Pat. No. 6,376,194, incorporated by reference herein in its entirety for all purposes. Solid phase affinity for nucleic acids according to the disclosed methods can be through any one of a number of means typically used to bind a solute to a substrate. Examples of such means include but are not limited to, ionic interactions (e.g., anion-exchange chromatography) and hydrophobic interactions (e.g., reversed-phase chromatography), pH differentials and changes, salt differentials and changes (e.g., concentration changes, use of chaotropic salts/agents). Exemplary pH based solid phases include but are not limited to those used in the INVITROGEN ChargeSwitch Normalized Buccal Kit magnetic beads, to which bind nucleic acids at low pH (<6.5) and releases nucleic acids at high pH (>8.5) and mono-amino-N-aminoethyl (MANAE) which binds nucleic acids at a pH of less than 7.5 and release nucleic acids at a pH of greater than 8. Exemplary ion exchange based substrates include but are not limited to DEA-SEPHAROSE™, Q-SEPHAROSE™, and DEAE-SEPHADEX™ from PHARMACIA (Piscataway, N.J.), DOWEX® I from The Dow Chemical Company (Midland, Mich.), AMBERLITE® from Rolun & Haas (Philadelphia, Pa.), DUOLITE® from Duolite International, In. (Cleveland, Ohio), DIALON TI and DIALON TIL.

The information contained in RNA in a sample can be converted to cDNA by using reverse transcription using techniques well known to those of ordinary skill in the art (see e.g., Sambrook et al, supra). PolyA primers, random primers, and/or gene specific primers can be used in reverse transcription reactions. In some embodiments, polyA primers, random primers, and/or gene specific primers are employed in reverse transcription reactions in the presently described methods.

The cDNA of the present invention is prepared using any conventional methods for preparing cDNA. The standard method for preparing cDNA from mRNA is by reverse transcription-PCR. Reverse transcription-PCR (often referred to as RT-PCR) is a well-known technique that is regularly employed by those of skill in the art to convert mRNA into DNA and a variety of references are available and provide detailed protocols.

Additionally, a number of template dependent processes are available to amplify primer sequences present in a given template sample. One of the best known amplification methods is the polymerase chain reaction (referred to herein as PCR) which is described in detail in U.S. Pat. Nos. 4,683,195, 4,683,202 and 4,800,159, and in Innis et al., 1990, each of which is incorporated herein by reference in its entirety. Related methods of amplification that may be advantageously employed include real-time PCR, quantitative real-time PCR, digital PCR (dPCR), digital emulsion PCR (dePCR), clonal PCR, amplified fragment length polymorphism PCR (AFLP PCR), allele specific PCR, assembly PCR, asymmetric PCR (in which a great excess of primers for a chosen strand is used), colony PCR, helicase-dependent amplification (HDA), Hot Start PCR, inverse PCR (IPCR), in situ PCR, long PCR (extension of DNA greater than about 5 kilobases), multiplex PCR, nested PCR (uses more than one pair of primers), single-cell PCR, touchdown PCR, loop-mediated isothermal PCR (LAMP), and nucleic acid sequence based amplification (NASBA). Other amplification schemes include: Ligase Chain Reaction (LCR), Branch DNA Amplification, Rolling Circle Amplification, Circle to Circle Amplification and other cyclical synthesis of single-stranded and double-stranded DNA, SPIA amplification, Target Amplification by Capture and Ligation (TACL) and other PCR-like template- and enzyme-dependent synthesis using primers with a capture or detector moiety, RACE amplification, Qbeta Replicase, isothermal amplification, strand displacement amplification (SDA), transcription-based amplification systems (TAS), and di-oligonucleotide amplification.

Suitable nucleic acid amplification conditions include well known parameters, such as: time, temperature, pH, buffers, reagents, cations, salts, co-factors, nucleotides, nucleic acids, and enzymes. In some embodiments, a PCR reaction includes ATP and/or NAD. In some embodiments, a reagent or buffer includes a source of ions, such as KCl, K-acetate, NH₄-acetate, K-glutamate, NH₄Cl, or ammonium sulfate. In some embodiments, a reagent or buffer includes a source of ions, such as magnesium, manganese, cobalt, or calcium. In some embodiments, a reagent or buffer includes acetate or chloride. In some embodiments, a buffer can include Tris, Tricine, HEPES, MOPS, ACES, MES, or inorganic buffers such as phosphate or acetate-based buffers which can provide a pH range of about 4-12. In some embodiments, a buffer includes chelating agents such as EDTA or EGTA. In some embodiments, a buffer includes dithiothreitol (DTT), glycerol, spermidine, BSA (bovine serum albumin) and/or Tween.

Polymerases that can be used for amplification in the methods of the provided invention include, for example, Taq polymerase, AccuPrime polymerase, or Pfu. The choice of polymerase to be used in the methods described herein can be based on whether fidelity or efficiency is preferred. In some embodiments, a high fidelity polymerase is employed.

Conventional techniques for mRNA profiling include Northern hybridization, cloning, and microarray analysis. (Wang, Ach and Curry. 2007. Direct and sensitive miRNA profiling from low-input total RNA. RNA 13(1): 151-9, Wang and Cheng. 2008. A simple method for profiling miRNA expression. Methods Mol Biol 414: 183-90, Shingara, Keiger, Shelton, Laosinchai-Wolf, Powers, Conrad, Brown and Labourier. 2005. An optimized isolation and labeling platform for accurate microRNA expression profiling. RNA 11(9): 1461-70, Nelson, Baldwin, Scearce, Oberholtzer, Tobias and Mourelatos. 2004. Microarray-based, high-throughput gene expression profiling of microRNAs. Nat Methods 1(2): 155-61).

Any combination of the above can be employed by one of skill, as well as combined with other known and routine methods, and such combinations are contemplated by the present invention.

In some embodiments, primers are tested and designed in a laboratory setting. In some embodiments, primers are designed by computer based in silico methods. Primer sequences are based on the sequence of the amplicon or target nucleic acid sequence that is to be amplified. Shorter amplicons typically replicate more efficiently and lead to more efficient amplification as compared to longer amplicons. Those of skill in the art are well aware of the basics regarding primer design for a target nucleic acid sequence and a variety of reference manuals and texts have extensive teachings on such methods, including for example, Molecular Cloning and Current Protocols, supra; the PrimerAnalyser Java tool available on the World Wide Web at primerdigital.com/tools/PrimerAnalyser.html and Kalendar et al. (Genomics, 98(2): 137-144 (2011)).

In specific embodiments, the sequencing step comprises the nested PCR technique described in International PCT Application No. PCT/US15/62018, wherein primers directed to the constant (C) region along with universal primers are preferably employed to capture TCR mRNAs in fragmented mRNA. Alternatively, the sequencing step comprises the nested PCR technique described in U.S. Patent Publication 2016/0034637, wherein primers directed to the variable (V) region and to the joining (J) or C region are preferably employed. More conventional primer strategies can also be used.

The present method of the present invention can be performed using mRNA isolated from any of a variety of biological samples containing T-cells. Methods for obtaining such samples are well-known to those of skill in the art and any appropriate methods can be employed to obtain samples containing or believed to contain T-cells. Biological samples may be stored if care is taken to reduce degradation, e.g. under nitrogen, frozen, or a combination thereof. The volume of sample used is sufficient to allow for measurable detection, for example from about 0.1, ml to 1 ml of a biological sample can be sufficient.

Biological samples for use in the methods provided in the present disclosure include, for example, a bodily fluid from a subject, including amniotic fluid (surrounding a fetus), aqueous humor, bile, blood and blood plasma, cerumen (earwax), Cowper's fluid or pre-ejaculatory fluid, chyle, chyme, female ejaculate, interstitial fluid, lymph, menses, breast milk, mucus (including snot and phlegm), pleural fluid, pus, saliva, sebum (skin oil), semen, serum, sweat, tears, urine, vaginal secretions, vomit, feces, internal body fluids including cerebrospinal fluid surrounding the brain and the spinal cord, synovial fluid surrounding bone joints, intracellular fluid (the fluid inside cells), and vitreous humour (the fluids in the eyeball. Biological sample contemplated by the disclosure also include biopsy samples from for example infection sites, cancer tissue or other diseased or potentially diseased tissue.

In some embodiments, the said biological sample is a body fluid sample and/or tissue sample. In some embodiments, the biological sample is selected from the group consisting of blood, plasma, serum, bone marrow, semen, vaginal secretions, urine, amniotic fluid, cerebrospinal fluid, synovial fluid and biopsy tissue samples, including from infection and/or tumor locations.

Diseased or infected tissues can be obtained from subjects with a wide variety of disease and disorders. Such disease and disorders include cancer, inflammatory diseases, autoimmune diseases, allergies and infections of an organism. The organism is preferably a human subject but can also be derived from non-human subjects, e.g., non-human mammals. Examples of non-human mammals include, but are not limited to, non-human primates (e.g., apes, monkeys, gorillas), rodents (e.g., mice, rats), cows, pigs, sheep, horses, dogs, cats, or rabbits.

Examples of cancer include prostrate, pancreas, colon, brain, lung, breast, bone, and skin cancers.

Examples of inflammatory conditions include irritable bowel syndrome, ulcerative colitis, appendicitis, tonsilitis, and dermatitis.

Examples of atopic conditions include allergy, asthma, etc.

Examples of autoimmune diseases include IDDM, RA, MS, SLE, Crohn's disease, Graves' disease, etc. Autoimmune diseases also include Celiac disease, and dermatitis herpetiformis. For example, determination of an immune response to cancer antigens, autoantigens, pathogenic antigens, vaccine antigens, and the like is of interest.

Examples of infections can include viral, fungal and bacterial, as well as antibiotic resistant bacterial infections. Examples of viral infections include influenza, cytomegalovirus (CMV), RSV, influenza virus, herpes simplex virus type 1, and parainfluenza virus. Examples of fungal infections include Aspergillus (e.g., A. fumigatus) or Candida (e.g., Candida albicans), and which may or may not exhibit resistance to antibiotic treatments. Examples of bacterial infections include Lysteria monocytogenes, Pseudomonas sp. (e.g., P. aeruginosa), Serratia marcescens, Clostridium difficile, Staphylococcus aureus, Staphylococcus sp., Acinetobacter sp., Enterococcus sp., Enterobacteria sp., E. coli, Klebsiella sp., Streptococcus (e.g., S. pneumoniae), Haenmophilus influenzae, and Neisseria meningitidis. Examples of drug resistant or multi-drug resistant microorganisms include, Staphylococcus aureus, Enterococcus sp., Pseudomonas sp., Klebsiella sp., E. coli, and/or Clostridium Difficile. Examples of drug-resistant microorganisms include methicillin-resistant or vancomycin-resistant Staphylococcus aureus (MRSA or VRSA) including intermediate resistant isolates, and carbapenum-resistant E. coli, Klebsiella, or Pseudomonas including intermediate resistant isolates.

In some embodiments, samples including or believed to include T-cells are obtained from an organism after the organism has been challenged with an antigen (e.g., vaccinated). In other cases, the samples are obtained from an organism before the organism has been challenged with an antigen (e.g., vaccinated). Comparing the diversity of the T-cell receptor repertoire present before and after challenge, can assist the analysis of the organism's response to the challenge.

The TCR α and β chain pairs identified by the subject methods may possess at least one desired functional property such as their affinity, avidity, cytolytic activity and the like, and can be advantageously utilized in a variety of subsequent therapeutic and diagnostic indications. For example, TCR α and β chain pairs identified as having desirable anti-tumor or anti-infective activity can be cloned into artificial T cell receptors for use in adoptive cell transfer protocols such as, e.g., CAR-T cell therapy. Similarly, knowledge of these pairings can be utilized to develop markers for, e.g., disease staging and/or treatment. Alternatively, TCR α and β chain pairs identified as having potential autoimmune effects can be employed in protective immune settings, e.g., as part of T cell vaccine strategies, and/or also be exploited for their potential diagnostic value.

In some embodiments, the methods are employed in order to optimize therapy, for example by analyzing the TCR α and β chain pairs in a sample, and based on that information, selecting the appropriate therapy, dose, treatment modality, etc. that is optimal for stimulating or suppressing a targeted immune response, while minimizing undesirable toxicity. The treatment is optimized by selection for a treatment that minimizes undesirable toxicity, while providing for effective activity. For example, an organism may be assessed for the TCR α and β chain pairs relevant to an autoimmune disease, and a systemic or targeted immunosuppressive regimen may be selected based on that information.

The identification of particular TCR α and β chain pairs in a subject can indicate the presence of a condition of interest. For example a history of cancer (or a specific type of allergy) can be reflected in the particular TCR α and β chain pairs identified in a given subject. Similarly, the presence of autoimmune disease may be reflected in the presence of particular TCR α and β chain pairs that bind to autoantigens. A signature can be obtained from all or a part of a dataset obtained by the methods of the present invention, usually a signature will comprise repertoire information from at least about 20 different TCR α and β chain pairs, at least about 50 different TCR α and β chain pairs, at least about 100 different TCR α and β chain pairs, at least about 10² different TCR α and β chain pairs, at least about 10³ different TCR α and β chain pairs, at least about 10⁴ different TCR α and β chain pairs, at least about 10⁵ different TCR α and β chain pairs, or more. Where a subset of the dataset is used, the subset may comprise, for example, alpha TCR or beta TCR, or a combination thereof.

Also provided are reagents and kits thereof for practicing one or more of the above-described methods. The subject reagents and kits thereof may vary greatly and can include any of the reagents and components described herein, including in particular reagents specifically designed for use in the subject methods. For example, the kits of the subject invention can include the specific primers provided in the examples below. The kits can further include a software package for statistical analysis, and may include a reference database for collecting and/or correlating the various TCR α/γ and β/δ chain pairs with specific diseases, disorders or infections for subsequent therapeutic and/or diagnostic use.

The kit may include reagents employed in the various methods, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, DNA kinase, DNA ligases and the like, various buffer mediums, e.g. hybridization and washing buffers, ligation buffers, and components, like spin columns, etc.

In addition to the above components, the subject kits will further include instructions for practicing the subject methods. These instructions may be present in the subject kits in a variety of forms, one or more of which may be present in the kit, and which include a printed and/or or computer readable format.

The following examples are provided for purposes of illustration, not limitation.

### EXAMPLES

### Example 1: Determination of Alpha and Beta Pairing

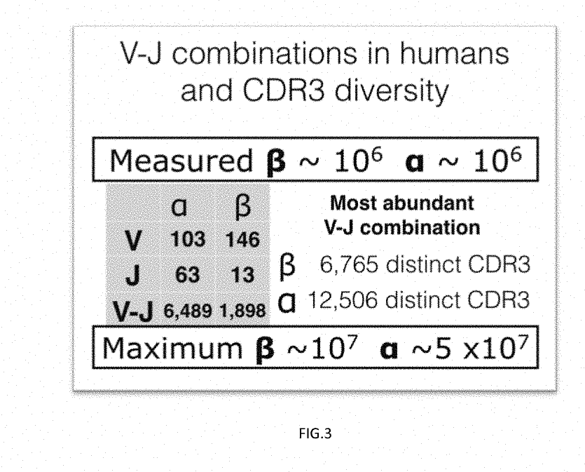

FIG. 3 shows data from TCR repertoire sequencing carried out on RNA isolated from 2 mL of human blood. At the top we list the number of distinct α and β sequences measured in our experiment. The figure shows the number of distinct V and J segments of each type and the number of distinct combinations that are possible (6489 V-J combinations for a and 1898 combinations for β). The machinery that generates the CDR3 diversity in each VJ pairing is the same, so we can infer the maximum possible diversity, shown at the bottom, assuming each combination of V-J can have, utmost, the same amount of diversity as the dominant VJ combination.

There are 0.54-1.79×10⁶ T cells in a mL of human blood. We measured, independently for α and β, a million different TCR sequences each, from approximately 2 mL of blood. If the million α and β were independently associating with each other, we would expect over 10¹² combinations, which could never be fully characterized from ˜million cells. This suggests that the α-β combinations are not independently associating with each other, instead there are a restricted set of α-β combinations. Thus, sorting the cells by α CDR3 of interest and then sequencing the β (or vice versa), can help to more efficiently identify TCR clones of value in diagnostics and therapeutics. The invention, therefore, comprises characterizing the α and β repertoires independently, identifying potentially interesting CDR3 in the α and β, sorting the T cells by the CDR3 (say α) and sequencing the counterpart (β) CDR3 to identify TCR clones of interest.

### Materials and Methods:

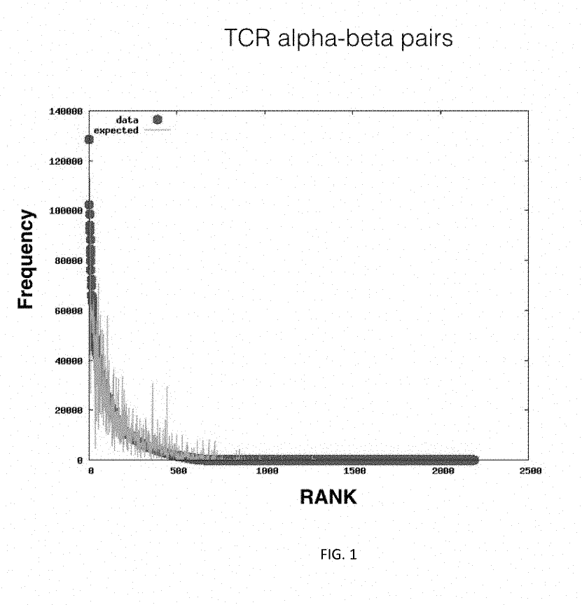

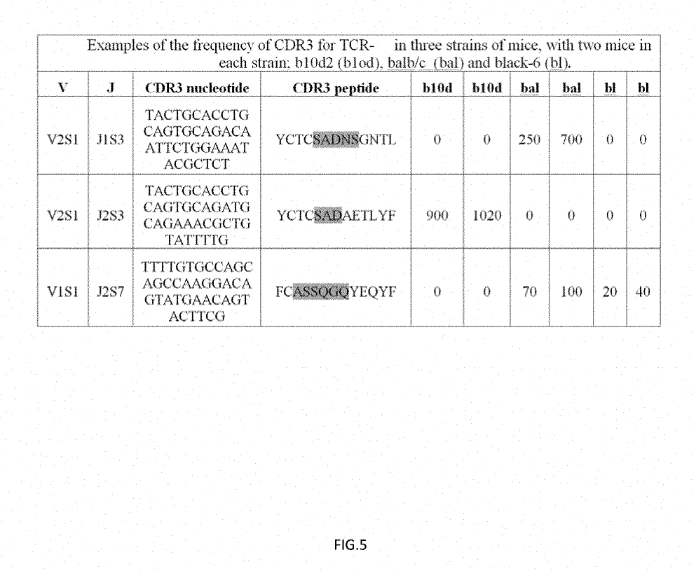

2 ml blood from an adult female was taken in EDTA tubes, and total RNA was isolated using AllPrep Universal kit using columns (cat #80224 from qiagen). The overall protocol is shown in FIG. 1. The RNA quality and quantity was checked using an Agilent Bioanalzyer. mRNA was isolated, fragmented, converted to cDNA, and ligated using adapters (5130-01 NEXTflex qRNA-seq, Bioo Scientific) that contained molecular indexing (see FIG. 5), following the manufacturer's instructions. The 5130-01 kit provides a mix of 96 different adapters to be able to measure clonality. Two rounds of per using the following TCR-specific primers gave a TCR rich library sequenced on the Illumina platform (30 bp Read 1 and 120 bp Read 2).
Reagents for Fragmentation, first strand, second strand, end repair, A-base addition, adapter ligation (5130-01 NEXTflex qRNA-seq, Bioo Scientific)
Ampure beads (Beckman coulter, Agencourt, AMPure XP—PCR Purification, Item No. A63880)
Primers for PCR 1 and 2 (self designed and synthesized by IDT):

The primers used to isolate α TCR sequences in PCR 1 and PCR 2 were self-designed and synthesized by IDT as follows:

Primers Used to Isolate Alpha TCR Sequences in PCR 1 for Human:

F = universal adapter:
(SEQ ID NO: 1)
AATGATACGGCGACCACCGAGATCTACACTCTTTCCCTACACGACGCTCT
TCCGATC*T
R = TCR C alpha primer (human):
(SEQ ID NO: 2)
CACTGGATTTAGAGTCTCTCAGC

F = universal adapter
(SEQ ID NO: 1)
AATGATACGGCGACCACCGAGATCTACACTCTTTCCCTACACGACGCTCT
TCCGATC*T
R = TCR C alpha primer (human)
(SEQ ID NO: 3)
CAAGCAGAAGACGGCATACGAGAT[TGGTCA]GTGACTGGAGTTCAGACG
TGTGCTCTTCCGATCTNNNGCTGGTACACGGCAGGGTCA

F = univeisal adapter
(SEQ ID NO: 1)
AATGATACGCCGACCACCGAGATCTACACTCTTTCCCTACACGACGCTCT
TCCGATC*T
R = TCR C beta primer (human)
(SEQ ID NO: 4)
TGCTTCTGATGGCTCAAACA

Primers Used to Isolate Beta TCR Sequences in PCR 1 for Human:

F = universal adapter
(SEQ ID NO: 1)
AATGATACGGCGACCACCGAGATCTACACTCTTTCCCTACACGACGCTCT
TCCGATC*T
R = TCR C beta primer (human)
(SEQ ID NO: 5)
CAAGCAGAAGACGGCATACGAGAT[CACTGT]GTGACTGGAGTTCAGACG
TGTGCTCTTCCGATCTNNNNNNCAGCGACCTCGGGTGGGAAC

Total RNA was isolated from cells and mRNA was pulled down from the total RNA (using poly-T beads). The mRNA was then fragmented (can be either by chemical means or by sonication), and cDNA was synthesized from the fragments using random hexamers as primers. The cDNA was end-repaired to get blunt ends and universal adapters, Adapter1 and Adapter2 (compatible with the sequencing platform), were ligated to the cDNA fragments. Up to this point the preparation followed standard mRNA-seq protocol. At this point a nested PCR was carried out using Adapter1 an internal primer (C-P1) which is complementary to a section of the C segment. This selects cDNA fragments that contain C. After this, a second PCR was carried out using Adapter1 and a new adapter, C-P2 that is complementary to a fragment in the portion of the C that remains, but contains additionally Adapter2 on the 5′ end. This second PCR generates a product that can be sequenced. A size-selection step ensures that the fragment is of sufficient length, (>200 nt) to span the CDR3 and contain enough of the V-segment allowing recognition of the V, J and the CDR3 sequences from the sequencing data.

Our methods of sequence analysis: We designed a new pipeline, tailored to our method, to measure usage of various segments and identify novel segments or combinations (such as alternative use of a leader sequence with different V segments). We used annotations compiled from the IMGT and EST databases to create non-redundant TCR-segment sequences, which we grouped into sets of Vs, Js, Ds and Cs. Using BLAST in sensitive settings (blastn, word size W set at 7), we mapped the sequences from our experiments to the non-redundant set, and for each read, identified Vs, Ds, Js and Cs.

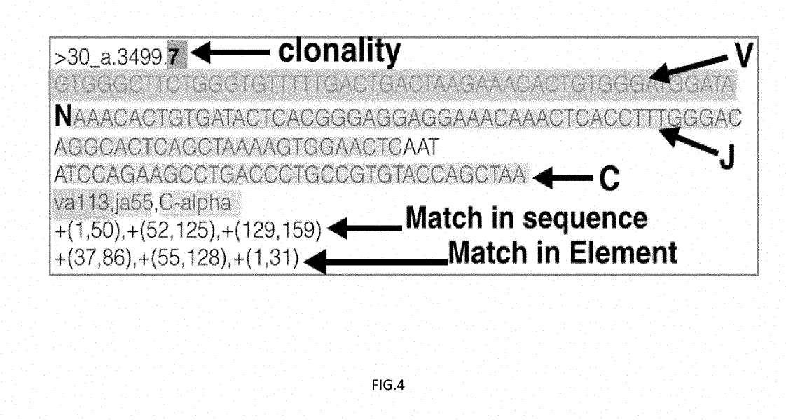

FIG. 4 shows the output of the analytical pipeline, which tracks the clonality and the various annotations (or lack thereof). It is a fasta-like formatted table; the first line consists of the name and a number showing clonality of the read. The next line is the composite read, the row after that gives the various annotations (V, J and C) and the last two rows are the matches from the composite read to matches on the corresponding V/J/C elements. This allows identification of the CDR3, helps identify novel segment usage and helps better annotate the TCR loci.

Cdr3 Curation:

The amino-acid sequences of the CDR3s are identified by comparing the translations of the three frames of the mRNA sequence with 3′ terminal amino acids of the preceding V segment and 5′ terminal amino acids of the subsequent J segments. Motifs for the ends of V's and J's are known (and can be inferred from our data based on frequency of occurrence) (FIG. 5).

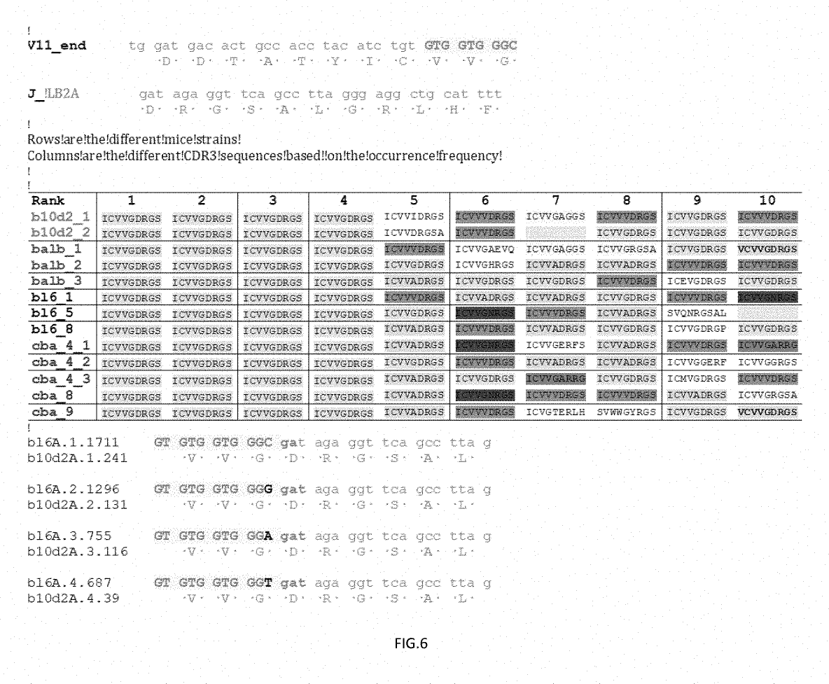

The sequences of the end of a particular V and the start of a particular J are shown in FIG. 6. The middle section of FIG. 6 shows the peptide sequence from top 10 different CDR3 nucleotide sequences made by the selected VJ combination. Different mouse strains are in different rows. Columns are different CDR3 sequences based on occurrence frequency. We observe that the first four-peptide sequence is the same in all mice strains independent of the nucleotide sequence. The bottom panel lists the nucleotide sequence and the frequency of this top CDR3 peptide sequence of two mice strains.

From FIG. 6 we see that for a particular VJ combination the dominant amino-acid sequences are the same across strains. What is surprising is the lack of diversity at the amino-acid level, even though we expected a large number of mutations. It is possible that this may change under infection or some other perturbation of the TCR repertoire.

### Example 2: Determination of Human Alpha and Beta Pairing from Large Sample Sizes

Normal human blood samples were obtained. The samples were donated to the NY blood center (NYBC) by a diverse set of humans, representing a cross-section of people classified by ethnic groups, gender and age. The aim of the human study is to identify the “normal” repertoire in healthy individuals and the possible role of various factors such as age, gender and ethnic origins on the repertoires. TCR repertoires (CDR3) for alpha and beta chains were sequenced from over 100 human samples. For 25 samples, CDR3 sequence data was sorted and analyzed separately for CD4+ and CD8+ T cells.

In order to analyze the samples CDR3, and V and J segments were identified, as well as boundaries regions between extracellular and intracellular segments. The different segments (e.g., CDR3, V, J) were grouped for α and β TCR chains and analyzed to identify αβ pairing patterns and CDR3 sequence frequency patterns indicative of normal samples.

### Example 3: Determination of Alpha and Beta Pairing from Mice Treated with Cancer Therapeutics

Mouse samples from mice genetically prone to tumors, some of whom were treated with cancer immunotherapy drugs (anti CTLA-4, anti PD-1 and a combination of the two) were obtained to study the effect of the treatment on the TCR repertoires. The samples were generated in the laboratory of Lawrence Fong, at UCSF. Lymph node samples were collected as well as tumor samples, if available, and the repertoires between these mice were compared (a total of 96 mice were in the cohort).

Candidate β-TCR chain CDR3 sequences indicative of immunotherapy treatments in mice were identified. The oligonucleotides in Table 1 have been designed and synthesized for labeling and sorting and then sequencing of cells having β-TCR chain CDR3 sequences of interest to determine the corresponding α-TCR chain CDR3 sequences.

| | | | | 1_ | 1_1 | | 1_1 | |
|---|---|---|---|---|---|---|---|---|
| V | J | CDR3 | Oligo | 1 | 0 | 1_11 | 2 | 1_13 |
| vb3 | jb5 | ASSPGQDN | GCCAGCTCTCCGGGACAGGATAAT | 23 | 135 | 3941 | 12 | 3764 |
| 3 | | (SEQ ID NO: 6) | (SEQ ID NO: 15) | 5 | 2 | 1 | | 6 |
| vb7 | jb1 | AWGQGGN | GCCTGGGGACAGGGAGGCAAC | 17 | 434 | 2346 | 6 | 3238 |
| 3 | | (SEQ ID NO: 7) | (SEQ ID NO: 16) | 4 | | 9 | | 1 |
| vb7 | jb14 | AWSPSGTGGR | GCCTGGAGTCCTTCCGGGACTGGGGGGAGG | 0 | 0 | 5783 | 38 | 2886 |
| 3 | | (SEQ ID NO: 8) | (SEQ ID NO: 17) | | | 5 | | 2 |
| vb3 | jb12 | ASSLQSS | GCAAGCAGCTTGCAGTCTAGT | 22 | 141 | 2894 | 25 | 2688 |
| 9 | | (SEQ ID NO: 9) | (SEQ ID NO: 18) | 7 | 3 | 3 | | 4 |
| vb4 | jb11 | ASSLVWEKQ | GCCAGCAGTTTGGTCTGGGAGAAGCAA | 23 | 113 | 2427 | 28 | 2297 |
| 4 | | (SEQ ID NO: 10) | (SEQ ID NO: 19) | 5 | 9 | 9 | | 5 |
| vb3 | jb11 | ASGG | GCCAGCGGTGGA | 57 | 149 | 2370 | 28 | 2426 |
| 5 | | (SEQ ID NO: 11) | (SEQ ID NO: 20) | 3 | 1 | 2 | | 1 |
| vb3 | jb10 | ASSFGATS | GCCAGCAGTTTCGGGGCAACTAGT | 20 | 138 | 2423 | 32 | 3007 |
| 9 | | (SEQ ID NO: 12) | (SEQ ID NO: 21) | 1 | 1 | 2 | | 2 |
| vb3 | jb3 | ASSLGQGN | GCCAGCTCTCTAGGACAGGGGAAC | 12 | 883 | 1912 | 25 | 2064 |
| 3 | | (SEQ ID NO: 13) | (SEQ ID NO: 22) | 9 | | 2 | | 9 |
| vb2 | jb1_ | ASSLGVA | GCCAGCTCTCTCGGTGTTGGA | 12 | 896 | 1655 | 18 | 1767 |
| 9 | 2 | (SEQ ID NO: 14) | (SEQ ID NO: 23) | 2 | | 8 | | 5 |

All of the U.S. patents, U.S. patent application publications, U.S. patent applications, foreign patents, foreign patent applications and non-patent publications referred to in this specification are incorporated herein by reference, in their entirety to the extent not inconsistent with the present description.

From the foregoing it will be appreciated that, although specific embodiments described herein have been described herein for purposes of illustration, various modifications may be made without deviating from the spirit and scope described herein. Accordingly, the disclosure is not limited except as by the appended claims.

What is claimed is:

1. A method for identifying T cell receptor chain pairings in T cell(s) of interest within a population of T cells, comprising:
a) contacting said population of T cells with a plurality of probes directed to either the TCR α/γ chain or the TCR β/δ chain in the T cell(s) of interest, wherein said probes target at least a portion of the variable domain of the individual receptor chain;
b) sorting said population of T cells based on binding of said plurality of probes to select for said T cell(s) of interest; and
c) sequencing the counterpart TCR β/δ chain or TCR α/γ chain, respectively, in the sorted T cell(s) of interest.

2. The method according to claim 1, wherein the probes target at least a portion of the complementarity determining region of the individual chain.

3. The method according to claim 2, wherein the probes target at least a portion of the CDR3.

4. The method according to claim 1, wherein said probes are nucleic acid probes

5. The method according to claim 4, wherein said probes are RNA probes.

6. The method according to claim 1 wherein said sorting step comprises flow cytometry.

7. The method according to claim 6, wherein said sorting step comprises live cell RNA detection.

8. The method of claim 1, further comprising isolating mRNA from said selected T cell(s) of interest, and contacting said isolated mRNA from said T cell(s) with said plurality of probes.

9. The method according to claim 1, wherein said sequencing step comprises a nested PCR reaction on fragmented mRNA from said selected T cell(s) of interest.

10. The method according to claim 9, wherein said nested PCR reaction comprises constant (C) region primers.

11. The method according to claim 9, wherein said nested PCR reaction comprises variable (V) region primers.

12. The method according to claim 1, comprising an initial step of obtaining a sample comprising said population of T cells from a subject.

13. The method according to claim 12, wherein said subject is tumor-bearing, and said T cells are tumor-infiltrating lymphocytes.

14. The method according to claim 12, wherein said subject is suffering from an infection.

15. The method according to claim 12, wherein said subject is suffering from an allergic condition.

16. The method according to claim 12, wherein said subject is suffering from an autoimmune disorder.

17. The method according to claim 12, wherein said subject is human.

18. A method for identifying heavy and light chain pairings in a B cell(s) of interest within a population of B cells, comprising:
a) contacting said population of B cells with a plurality of probes directed to either the heavy chain or light chain in the B cell(s) of interest, wherein said probes target at least a portion of the variable domain of the individual chain;
b) sorting said population of B cells based on binding of said plurality of probes to select for said B cell(s) of interest; and
c) sequencing the counterpart light chain or heavy chain, respectively, in the sorted B cell(s).
